(19) United States
(12) Patent Application Publication
(10) Pub. No.: US 2024/0350572 A1
(43) Pub. Date: Oct. 24, 2024

(54) ENHANCED CANNABINOID COMPOSITIONS AND METHODS FOR MAKING SAME

(71) Applicants: CRISP Curtis, LAMESA, TX (US); TAAC Naturals, LLC, Los Angeles, CA (US)

(72) Inventors: Curtis Crisp, Lamesa, TX (US); Allen Cagle, Los Angeles, CA (US)

(21) Appl. No.: 18/577,907

(22) PCT Filed: Jul. 8, 2022
(86) PCT No.: PCT/US2022/073543

§ 371 Date: Jan. 9, 2024

Related U.S. Application Data
(60) Provisional application No. 63/220,037, filed on Jul. 9, 2021.

(51) Int. Cl. A61K 36/185; B01D 11/02

(52) U.S. Cl. CPC A61K 36/3482; B01D 11/0288

(57) ABSTRACT

Enhanced cannabinoid compositions and methods for producing enhanced cannabinoid compositions are provided. The method can include winterizing a Cannabis extract. wherein the winterizing comprises mixing the Cannabis extract with a solvent at a temperature below −20 C; permitting separation of components of the extract for a period of time sufficient for an insoluble lipid fraction to separate from soluble components; collecting the insoluble lipid fraction; refining the lipid fraction; and/or adding an amount of refined lipid fraction to the composition; The amount of refined lipid fraction can be sufficient to result in an enhanced characteristic of the composition.

### CLAIM OF PRIORITY UNDER 35 U.S.C. § 119

The present Application for Patent claims priority to Provisional Application No. 63/220,037 entitled “ENHANCED CANNABINOID COMPOSITIONS AND METHODS FOR MAKING SAME” filed Jul. 9, 2021, which is hereby expressly incorporated by reference herein.

### BACKGROUND

### Field

This invention relates to enhanced cannabinoid compositions and methods for producing the same.

### Background

The genus Cannabis has been in use by humans for millennia, due to the multiplicity of its benefits to humans, including the considerable value and utility of its fiber, the nutritional value of its seeds, and the medicinal value of its floral parts and products made from them. Cannabis plants include secondary metabolites called cannabinoids. Cannabinoids are hydrophobic and can act on cannabinoid receptors in tissues and cells of the human body. Cannabidiol (CBD) is just one of at least dozens—perhaps hundreds—of cannabinoids endogenous to Cannabis. CBD extracted from Cannabis is widely used in over-the-counter medicines and topical treatments, and is also the active ingredient in the FDA-approved drug Epidiolex. The cannabinoids as a group interact with the human endocannabinoid receptors, which are distributed in the brain and throughout the body. The study of the endocannabinoid system (ECS) in humans and other mammals is an area of increasing interest and holds tremendous promise for the future of medicine. See, e.g., Russo (2019). Cannabis and Pain, Pain Medicine, 20 (10): 1093/pm/pnz227; and Russo (2016). Clinical Endocannabinoid Deficiency Reconsidered: Current Research Supports the Theory in Migraine, Fibromyalgia, Irritable Bowel, and Other Treatment-Resistant Syndromes, Cannabis Cannabinoid Res. 1 (1): 154-165. In concert with other Cannabis biomolecules, cannabinoids interact with receptors in the human body to produce an “entourage effect” with an immense variety of possible physiological outcomes (Russo, 2019).

### SUMMARY

Some embodiments of the invention relate to a method of enhancing a composition. The method can include winterizing a Cannabis extract, wherein the winterizing comprises mixing the Cannabis extract with a solvent at a temperature below −20° C.; permitting separation of components of the extract for a period of time sufficient for an insoluble lipid fraction to separate from soluble components; collecting the insoluble lipid fraction; refining the lipid fraction; and/or adding an amount of refined lipid fraction to the composition; The amount of refined lipid fraction can be sufficient to result in an enhanced characteristic of the composition.

In some embodiments, the solvent can be ethanol.

In some embodiments, the temperature can be about −80° C.

In some embodiments, the composition can be a topical skin formulation.

In some embodiments, the enhanced characteristic can be improved absorption; stability; shelf life; half-life; bioavailability of an active agent; an improved effect; and/or the like.

In some embodiments, the improved effect can be improved aesthetic appearance of skin.

In some embodiments, the improved effect can be reduction in pore size; improvement in skin tone, radiance, clarity and/or tautness; promotion of anti-oxidant activity; improvement in skin firmness; improvement in skin plumpness; improvement in skin suppleness; improvement in skin softness; improvement in pro-collagen; improvement in collagen production; improvement in skin texture; promotion of re-texturization; improvement in skin barrier repair; improvement in skin barrier function; improvement in appearance of skin contours; restoration of skin luster; restoration of skin brightness; replenishment of essential nutrients; replenishment of constituents in the skin decreased by aging, replenishment of constituents in the skin decreased by menopause, improvement in communication among skin cells; increase in cell proliferation; increase in cell multiplication; increase in skin cell metabolism decreased by aging; increase in skin cell metabolism decreased by menopause; improvement in skin moisturization; promotion or cell turnover; acceleration of cell turnover; enhancement of skin thickness; reducing skin sensitivity; increase in skin elasticity; increase in skin resiliency; enhancement of exfoliation, with or without the use of alpha or beta hydroxy acids, keto acids or other exfoliants; and/or the like.

In some embodiments, the improved effect can be determined by visual and/or physical evidence.

In some embodiments, the improved effect can be selected from a feeling of tighter, less oily skin; application of the composition that leaves less of a film on the surface of the skin; and quicker absorption of the composition into the skin.

In some embodiments, the composition can be a medication, and the enhanced characteristic can be increased systemic exposure and/or bioavailability.

Some embodiments of the invention relate to a composition enhanced by any method disclosed herein.

### DETAILED DESCRIPTION

Methods for enhancing a composition comprising cannabinoids are provided. Enhanced compositions produced with the methods described herein are also provided.

Methods of the invention can include separating a lipid fraction from a Cannabis extract and adding the lipid fraction to a composition in an amount sufficient to result in an enhanced composition.

The Cannabis extract can be prepared by combining ground Cannabis biomass and an organic solvent, such as, for example, ethanol, butane, propane, or the like. Solubilization allows components from the Cannabis biomass, including but not limited to terpenes, cannabinoids, and lipids to collect in a liquid fraction, or “crude”. The plant material from the Cannabis biomass remains insoluble and is not part of the crude. Any strain of Cannabis can be used to obtain a ground Cannabis biomass. Desirable strains can be selected based upon their profile of cannabinoids, terpenes, lipids, or other solvent-extractable components.

The lipid fraction of the crude can be obtained by “winterizing” or any similar method known in the art. Winterization can be achieved by dissolving a non-polar substance (e.g., the cannabinoid extract) into a polar solvent (e.g., ethanol) and then cooling the mixture at sub-zero temperatures. Winterizing can include mixing a Cannabis extract with ethanol or another polar solvent at a temperature below 20° C. and, in some embodiments, around −80° C.

The crude can be diluted 10:1 with ethanol, preferably above 90% purity. Afterward, the ethanol-diluted crude can be cooled at sub-zero temperatures. This cooling separates waxes and lipids from the cannabinoid extract, by causes them to rise to the top of the mixture for easy separation from the liquid fraction such as, for example, by filtration.

An exemplary winterization method is described in U.S. Pat. No. 7,344,736, which is fully incorporated by reference herein. In some embodiments, “winterizing” refers to cooling a crude extract in order to precipitate one or more waxes from a crude Cannabis extract. In some embodiments, the crude extract can be cooled to a temperature of below 0° C. For example, the crude extract may be cooled to −2° C., −4° C., −6° C., −8° C., or −10° C., or lower. The precipitated wax may then be filtered from the cooled crude extract.

The method can include permitting separation of components of the extract for a period of time sufficient for an insoluble lipid fraction to separate from soluble components. The period of time can be about 12-72 hours. For example, the period of time can be about 12, 24, 36, 48, 60, or 72 hours, or more. In some embodiments, the method can include collecting the lipid fraction. Collection can include scraping the liquid fraction off the top of the soluble components and into a second container. As used herein, the lipid fraction, can refer to the lipid byproduct of winterization. The lipid fraction is typically discarded during extraction methods. The lipid fraction, a byproduct of winterization, can contain waxes and/or lipids. The invention relates to the finding that the lipid fraction can be important to the therapeutic value of the extract. This is because it is becoming recognized that the plant's therapeutic value may not be attributable to the major cannabinoids alone. “Major” cannabinoids can include THC and CBD while “minor” cannabinoids can include those such as cannabigerol (CBG), delta 8-tetrahydrocannabinol (Δ8-THC), cannabinol (CBN). The therapeutic value may lie in a synergistic effect between numerous chemical components present in the Cannabis plants. The plant is believed to contain over a hundred cannabinoids, and numerous secondary metabolites of Cannabis, terpenes, and terpenoids, all of which are hypothesized to contribute synergistically to the plant's therapeutic benefits. This interaction or synergy resulting from the complex mixture of compounds present within the Cannabis plant is known as the entourage effect, in which a mixture of compounds demonstrates greater therapeutic efficacy than any of its constituent compounds in isolation.

The invention relates to methods for capturing the non-cannabinoid portions of the extract that would assist in preserving the entourage effect, while removing plant matter that may detract from the stability or safety of the formulation during the extraction process. When breaking down the plant matter, the initial extract contains particulate materials, chlorophyll, large sugars (e.g., plant cellulose), and proteins that may not have the desired effects or impact on other aspects of the product, such as its shelf-life and stability. Thus, the invention uses extraction methods whereby undesirable plant materials are removed, but desirable waxes and lipids, which contribute to the entourage effect, are not discarded.

In some embodiments, the method can include refining the lipid fraction before adding it to a composition to result in an enhanced characteristic of the composition.

The refining of the lipid fraction can include cleaning the lipid fraction with a solvent. The solvent can be but is not limited to ethanol. This mixture can then be placed in a vacuum oven or a similar device to purge the solvents.

The vacuum oven can be at a temperature sufficient to evaporate the solvent but not burn the lipids. For example, the vacuum oven can be at a temperature of about 110° C. (e.g., 40° C., 60° C., 80° C., 106° C., 108° C., 110° C., 112° C., or 114° C., 120° C., 140° C., 160° C., 180° C., or 200° C.). During the refining process, the temperature can be raised slowly until the product begins to react. During this process, the product is monitored over a period of about 6-8 hours (e.g., 5.5 or less, 6, 6.5, 7, 7.5, 8, or 8.5, or more hours) as the temperature of the oven continues to increase. The increasing of the temperature is stopped once the product stops reacting. Reaction of the product can look like simmering. An indication of the reaction stopping (i.e., when the solvents have evaporated completely) can be when the simmering or movement of the product stops. In some embodiments, the product can be fully purged of solvents around about 200° C. (e.g., 192° C., 194° C., 196° C., 198° C., 200° C., 202° C., 204° C., 206° C., or 208° C.). When adding lipids to the composition, the composition becomes more stable at a lower temperature but melts and becomes fluid more easily as temperature increases.

As used herein, “improvement” can be defined as a change to a feature that enables it to become better. The term “improvement” refers to a change in a parameter in a desired direction. In some embodiments, a useful improvement is quantified by a 1% change in the measured feature. In other embodiments, the % change can be 2%, 3%, 5%, 10%, 20%, 30%, 40%, 50% or more.

As used herein, “increase” can be defined as becoming or making greater in size, amount, intensity, or degree, or any other parameter. In some embodiments, an increase can be quantified by a 1% increase change in the measured feature. In other embodiments, the % change can be 2%, 3%, 5%, 10%, 20%, 30%, 40%, 50% or more.

As used herein, “decrease” can be defined as becoming smaller or making fewer in size, amount, intensity, or degree or any other parameter. In some embodiments, a decrease can be quantified by a 1% decrease change in the measured feature. In other embodiments, the % change can be 2%, 3%, 5%, 10%, 20%, 30%, 40%, 50% or more.

As described herein, the method can include adding the lipid fraction derived from a Cannabis extract to a composition to enhance the composition. The enhanced composition can have an enhanced characteristic such as improved absorption; stability; shelf life; half-life; bioavailability of an active agent; improved effects (e.g., improved aesthetic appearance of skin); and/or the like. In some embodiments, in some formulations, the enhanced characteristic is improved texture, flavor, bioavailability, and/or the like.

For example, the composition can be a topical skin formulation and the effect can include improving the aesthetic appearance of skin. Improving the aesthetic appearance of the skin can include reduction in pore size; improvement in skin tone, radiance, clarity and/or tautness; promotion of anti-oxidant activity; improvement in skin firmness, plumpness, suppleness, and/or softness; improvement in pro-collagen and/or collagen production; improvement in skin texture and/or promotion of re-texturization; improvement in skin barrier repair and/or function; improvement in appearance of skin contours; restoration of skin luster and/or brightness; replenishment of essential nutrients and/or constituents in the skin decreased by aging, menopause, and/or the like; improvement in communication among skin cells; increase in cell proliferation and/or multiplication; increase in skin cell metabolism decreased by aging and/or menopause; improvement in skin moisturization; promotion and/or acceleration of cell turnover; enhancement of skin thickness; reducing skin sensitivity; increase in skin elasticity and/or resiliency; enhancement of exfoliation, with or without the use of alpha or beta hydroxy acids, keto acids or other exfoliants; and/or the like.

Absorption of the composition can be determined by visual and/or physical evidence. For example, visual evidence can include less shine and/or increased tightening of the skin. For example, physical evidence can include a feeling of tighter, less oily skin. Improved absorption can include application of the composition that leaves less of a “film” on the surface of the skin, for example, the enhanced composition absorbs into the skin quicker without leaving a film on the skin compared to an unenhanced composition; and/or the like

The composition can be a liquid or a cream capable of delivering an active agent such as a cannabinoid to a subject. The cannabinoid can be tetrahydrocannabinolic acid A (THCA-A), tetrahydrocannabinolic acid B (THCAB), tetrahydrocannabinol (THC), tetrahydrocannabinolic acid C (THCA-C), tetrahydrocannabinol C (THC-C), tetrahydrocannabivarinic acid (THCVA), tetrahydrocannabivarin (THCV), tetrahydrocannabiorcolic acid (THCA-C), tetrahydrocannabiorcol (THC-C), delta-7-cis-iso-tetrahydrocannabivarin, delta-8-tetrahydrocannabinolic acid (Δ8-THCA), delta-9-tetrahydrocannabinol (Δ9-THC), cannabidiolic Acid (CBDA), cannabidiol (CBD), cannabidiol monomethylether (CBDM), cannabidiol-C (CBD-C), cannabidivarinic acid (CBDVA), cannabidivarin (CBDV), cannabidiorcol (CBD-C), cannabigerolic acid (CBGA), cannabigerolic acid monomethylether (CBGAM), cannabigerol (CBG), cannabigerol monomethylether (CBGM), cannabigerovarinic Acid (CBGVA), cannabigerovarin (CBGV), cannabichromenic Acid (CBCA), cannabichromene (CBC), cannabichromevarinic Acid (CBCVA), cannabichromevarin (CBCV), cannabicyclolic acid (CBLA), cannabicyclol (CBL), cannabicyclovarin (CBLV), cannabielsoic acid A (CBEA-A), cannabielsoic acid B (CBEA-B), cannabielsoin (CBE), cannabinolic acid (CBNA), cannabinol (CBN), cannabinol methylether (CBNM), cannabinol-C4 (CBN-C4), cannabivarin (CBV), cannabinol-C (CBN-C), cannabiorcol (CBN-C1), cannabinodiol (CBND), cannabinodivarin (CBVD), cannabitriol (CBT), 10-Ethoxy-9-hydroxy-delta-6a-tetrahydrocannabinol, 8,9-dihydroxy-delta-6a-tetrahydrocannabinol (8,9-Di-OH-CBT-C5), cannabitriolvarin (CBTV), ethoxy-cannabitriolvarin (CBTVE), dehydrocannabifuran (DCBF), cannabifuran (CBF), cannabichromanon (CBCN), cannabicitran (CBT), 10-oxo-delta-6a-tetrahydrocannabinol (OTHC), delta-9-cis-tetrahydrocannabinol (Δ9-cis-THC), cannabiripsol (CBR), -3,4,5,6-tetrahydro-7-hydroxy-alpha-alpha-2-trimethyl-9-n-propyl-2,6-methano-2H-1-benzoxocin-5-methanol (OH-iso-HHCV), trihydroxy-delta-9-tetrahydrocannabinol (triOH-THC), an isocanabinoid, any other cannabinoid, and any combination thereof. For example, the composition can be a cream for topical use that contains one or more cannabinoids. For example, the composition can be any composition provided in Application No. PCT/US2021/044200 filed Aug. 2, 2021, which is fully incorporated by reference herein.

The composition can include amygdalin (also called nitriloside, purasin, and/or vitamin B17) or derivatives or variations thereof. For example, the composition can include Laetrile, a drug containing purified amygdalin. Amygdalin is found in many plants, such as in the seeds (kernels) of apricots, bitter almonds, apples, peaches, and plums. Thus, the composition can include extracts of plants containing amygdalin such as apricot seed extract. In some embodiments, apricot seed powder (ASP) with 50% amygdalin can be used. In other embodiments, percentage of amygdalin in the ASP can be greater or lower than 50% such as, for example, 1%, 2%, 5%, 10%, 20%, 30%, 40%, 60%, 70%, 80%, 90%, 95%, or 99%.

In some embodiments, the composition can be nano-emulsified. As used herein, “nano-emulsified” can refer to using an emulsifying agent to mix two immiscible liquids and form a single-phase liquid where the resulting singe-phase liquid contains emulsion structures less than 100 nm in diameter. In some embodiments, the immiscible liquids can be Cannabis oil and water. In some embodiments, the emulsifying agent can be a surfactant.

### Topical Formulation

In some embodiments, the composition can be a topical formulation. The topical formulation can include about 0.1%-90% lipid fraction (e.g., 0.1%, 1%, 5%, 10%, 20%, 30%, 40%, 50%, 60%, 70%, 80%, or 90%). In some embodiments, the volume of cream can include about 5% lipid fraction (e.g., 0.1%, 1%, 2%, 3% 4.5%, 5%, 5.5%, 6%, 7%, 8%, 10%, 15%, 20%, 25% or more). In some embodiments, such as a lip balm, the lip balm can include 0.1%-99% lipid fraction (e.g., 0.1%, 1%, 5%, 10%, 20%, 30%, 40%, 50%, 60%, 70%, 80%, 90%, 95%, or 99%).

Some embodiments of the invention relate to methods for producing an enhanced topical formulation. The method can include isolating a lipid fraction from a Cannabis extract; refining the lipid fraction; and adding the refined lipid fraction to a topical formulation to produce an enhanced topical formulation.

### Other formulations

It is a further object of the present invention to disclose an enhanced composition produced by any of the methods described herein. The composition can be formulated in a dosage form selected from the group consisting of liquid, solid, gas, oral, sublingual, pill, tablet, capsule, buccal, tincture, strip, film, spray, lozenge, effervescent form, sub-lingual, granules, orally-disintegrating, thin film, solution, suspension, emulsion, powder, solid crystals, paste, inhalational, aerosol, inhaler, nebulizer, smoking, vaporizer, parenteral, intradermal, intramuscular, intraosseous, intraperitoneal, intravenous, subcutaneous, topical, cream, gel, liniment or balm (such as a lip balm), shampoo, conditioner, lotion, ointment, drops, syrup, skin patch, vaginal, suppository, pessary, rectal and any combination thereof. The composition can include about 0.1%-90% lipid fraction or any amount sufficient to result in an enhanced composition.

In some embodiments the composition can be a medication or a pharmaceutical formulation and the enhanced characteristic is increased systemic exposure or bioavailability.

### EXAMPLES

### Example 1

The following example provides a method for enhancing a cream including CBD, B17, hyaluronic acid, castor oil, dimethyl sulfoxide (DMSO), collagen, and lanolin.
Heat cream to 125° F.
Heat lipid fraction to 125° F.
Combine 1-5% lipid fraction with 95-99% cream.
Let the mixture settle by allowing it to sit for at least 24 hours until it appears to have a matte finish and has solidified enough that tilting the container with the mixture does not cause the mixture to spill out.

### Example 2

The following example provides a method for enhancing cream (volume 3 oz) with 1500 mg CBD; 1500 mg B17; 5% of volume DSMO; 5% of volume lipids; cream base; 1% Vitamin A acetate.
Combine CBD, B17, DMSO, and the lipid fraction.
Nano-emulsify the composition with DSMO.
Add the remaining ingredients to form a cream formulation.

### Example 3

The enhanced creams of Examples 1 or 2 are compared to a cream without the added lipid fraction. The enhanced creams are found to have improved absorption. Cream absorption is improved by 1, 10, 25, or 50%, or more.

### Example 4

The enhanced creams of Examples 1 or 2 are compared to a cream without the added lipid fraction. The enhanced creams are found to have improved stability. Cream shelf-life is improved by 1, 10, 25, or 50%, or more.

### Example 5

The enhanced creams of Examples 1 or 2 are compared to a cream without the added lipid fraction. The enhanced creams are found to have improved effects. Cream effects are improved by 1, 10, 25, or 50%, or more.

The various methods and techniques described above provide a number of ways to carry out the application. Of course, it is to be understood that not necessarily all objectives or advantages described are achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some embodiments specifically include one, another, or several features, while others specifically exclude one, another, or several features, while still others mitigate a particular feature by including one, another, or several other features.

Furthermore, the skilled artisan will recognize the applicability of various features from different embodiments. Similarly, the various elements, features and steps discussed above, as well as other known equivalents for each such element, feature, or step, can be employed in various combinations by one of ordinary skill in this art to perform methods in accordance with the principles described herein. Among the various elements, features, and steps some will be specifically included, and others specifically excluded in diverse embodiments.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof.

In some embodiments, any numbers expressing quantities of ingredients, properties such as molecular weight, reaction conditions, and so forth, used to describe and claim certain embodiments of the disclosure are to be understood as being modified in some instances by the term “about.” Accordingly, in some embodiments, the numerical parameters set forth in the written description and any included claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are usually reported as precisely as practicable.

In some embodiments, the terms “a” and “an” and “the” and similar references used in the context of describing a particular embodiment of the application (especially in the context of certain claims) are construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. No language in the specification should be construed as indicating any non-claimed element essential to the practice of the application.

Variations on preferred embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting effect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

What is claimed is:

1. A method of enhancing a composition comprising:
a. winterizing a Cannabis extract, wherein the winterizing comprises mixing the Cannabis extract with a solvent at a temperature below −20° C.;
b. permitting separation of components of the extract for a period of time sufficient for an insoluble lipid fraction to separate from soluble components;
c. collecting the insoluble lipid fraction;
d. refining the lipid fraction;
e. adding an amount of refined lipid fraction to the composition;
wherein the amount of refined lipid fraction is sufficient to result in an enhanced characteristic of the composition.

2. The method of claim 1, wherein the solvent is ethanol.

3. The method of claim 1, wherein the temperature is about −80° C.

4. The method of claim 1, wherein the composition is a topical skin formulation.

5. The method of claim 4, wherein the enhanced characteristic is selected from at least one of the following group: improved absorption; stability; shelf life; half-life; bioavailability of an active agent; and an improved effect.

6. The method of claim 5, wherein the improved effect is improved aesthetic appearance of skin.

7. The method of claim 6, wherein the improved effect is selected from reduction in pore size; improvement in skin tone, radiance, clarity and/or tautness; promotion of anti-oxidant activity; improvement in skin firmness; improvement in skin plumpness; improvement in skin suppleness; improvement in skin softness; improvement in pro-collagen; improvement in collagen production; improvement in skin texture; promotion of re-texturization; improvement in skin barrier repair; improvement in skin barrier function; improvement in appearance of skin contours; restoration of skin luster; restoration of skin brightness; replenishment of essential nutrients; replenishment of constituents in the skin decreased by aging, replenishment of constituents in the skin decreased by menopause, improvement in communication among skin cells; increase in cell proliferation; increase in cell multiplication; increase in skin cell metabolism decreased by aging; increase in skin cell metabolism decreased by menopause; improvement in skin moisturization; promotion or cell turnover; acceleration of cell turnover; enhancement of skin thickness; reducing skin sensitivity; increase in skin elasticity; increase in skin resiliency; and enhancement of exfoliation, with or without the use of alpha or beta hydroxy acids, keto acids or other exfoliants.

8. The method of claim 5, wherein the improved effect is determined by visual and/or physical evidence.

9. The method of claim 8, wherein the improved effect is selected from a feeling of tighter, less oily skin; application of the composition that leaves less of a film on the surface of the skin; and quicker absorption of the composition into the skin.

10. The method of claim 1, wherein the composition is a medication and the enhanced characteristic is increased systemic exposure.

11. A composition enhanced by the method of claim 1.
